SEQUENCE LISTING (6 sequences)

SEQ ID NO: 1 - DNA, 46 bp, Artificial Sequence
  Chemically synthesized
tcatgtgctttgtatgaaacrccttggaatgctgataagtttaatt

SEQ ID NO: 2 - DNA, 51 bp, Artificial Sequence
  Chemically synthesized
gaatcacctattcyaaaggcgaatcygatcatgtggttcctgctgccctta

SEQ ID NO: 3 - DNA, 46 bp, Artificial Sequence
  Chemically synthesized
tcatgtgctttgtatgaaacgccttggaatgctgataagtttaatt

SEQ ID NO: 4 - DNA, 46 bp, Artificial Sequence
  Chemically synthesized
tcatgtgctttgtatgaaacaccttggaatgctgataagtttaatt

SEQ ID NO: 5 - DNA, 51 bp, Artificial Sequence
  Chemically synthesized
gaatcacctattcyaaaggcgaatctgatcatgtggttcctgctgccctta

SEQ ID NO: 6 - DNA, 51 bp, Artificial Sequence
  Chemically synthesized
gaatcacctattcyaaaggcgaatccgatcatgtggttcctgctgccctta

(19) United States
(12) Patent Application Publication
(10) Pub. No.: US 2009/0104598 A1
(43) Pub. Date: Apr. 23, 2009

(54) Dopamine D2 receptor gene variants

(75) Inventors: Rudi Hwang, Toronto (CA); James L. Kennedy, Toronto (CA)

Correspondence Address: WESTERMAN, HATTORI, DANIELS & ADRIAN, LLP, 1250 CONNECTICUT AVENUE, NW, SUITE 700, WASHINGTON, DC, 20036, US

(73) Assignee: CENTRE FOR ADDICTION AND MENTAL HEALTH, Toronto (CA)

(21) Appl. No.: 11/407,330

(22) Filed: Apr. 20, 2006

(51) Int. Cl. C12Q 1/68

(52) U.S. Cl. 435/6

(57) ABSTRACT

The present invention provides a method of predicting antipsychotic response to drug therapy comprising testing a sample obtained from a subject for the presence of a polymorphism in the dopamine D2 receptor gene DRD2, wherein the presence of the rs1079598T allele, the rs1125394A allele or both is predictive of the subject being susceptible to drug therapy.

### FIELD OF INVENTION

The present invention relates to gene variants predictive of drug treatment response.

### BACKGROUND OF THE INVENTION

Clinical response to clozapine treatment in schizophrenic patients is variable. It has been estimated that clozapine is an effective treatment for at least 70% of this patient population (Meltzer 1997). Dopamine (DA) D₂ receptor (D2) blockade is believed to be the central mechanism by which conventional antipsychotic drugs (APDs) achieve their effects (Carlsson 1978) and most likely plays a key role in response to newer atypical APDs such as clozapine (Meltzer 2003).

Previously, several DRD2 pharmacogenetic association studies have examined the −141C Ins/Del and TagIA SNPs. The Del allele of the −141C Ins/Del SNP has been associated with increased D₂ density levels in the human striatum (Jonsson et al. 1999; Pohjalainen et al. 1999) and therefore might also affect DRD2 expression in the mesocorticolimbic regions of human brain, regions believed to be involved in psychosis, cognition, and the negative symptoms of SCZ. Studies have associated the Del allele with greater improvement of positive symptoms in an ethnically diverse clozapine-treated sample (Malhotra et al. 1999) but not in a mixed-treatment Asian sample (Ohara et al. 1998).

No associations between the Del allele of the −141C Ins/Del SNP and negative symptom response have been reported. The A1 allele of the TagIA polymorphic site, on the other hand, has been associated with reduced D₂ density levels in the human striatum (Noble 1998; Pohjalainen et al. 1998; Ritchie and Noble 2003; Thompson et al. 1997). Recently this SNP has been shown to reside in the coding region of a novel kinase gene called ‘ankyrin repeat and kinase domain containing 1’ (ANKK1) and causes an amino acid substitution in the last ankyrin repeat (Neville et al. 2004; Dubertret et al. 2004). Pharmacogenetic studies have associated the A1 allele with positive symptom response in a haloperidol-treated Caucasian sample (Schafer et al. 2001) as well as a nemonapride-treated Asian sample (Suzuki et al. 2000), but not in a bromperidol-treated Asian sample (Suzuki et al. 2001). No associations have been reported between this SNP and negative symptom response, similar to the studies on the −141C Ins/Del marker.

### SUMMARY OF THE INVENTION

The present invention relates to gene variants predictive of drug treatment response.

According to the present invention there is provided a method of predicting antipsychotic response to drug therapy comprising,

testing a sample obtained from a subject for the presence of a polymorphism in the dopamine D2 receptor gene DRD2, wherein the presence of the rs1079598T allele, the rs1125394A allele or both is predictive of the subject being susceptible to drug therapy.

The present invention also provides a method as defined above, wherein the presence of the rs1079598T allele and the rs1125394A allele is predictive of the subject being susceptible to drug therapy.

Also provided by the present invention is a method as defined above wherein the subject is an African American subject.

In an alternate embodiment of the present invention, there is provided a method as defined above, wherein the sample is any biological sample from which DNA from the subject may be obtained. In a preferred embodiment, the sample is a blood sample.

Also encompassed by the present invention is a method as defined above, wherein the step of testing comprises DNA extraction and PCR analysis, sequencing, 5′exonuclease fluorescence assay, or a combination thereof. In a preferred embodiment, which is not meant to be limiting in any manner, the step of testing comprises a 5′ exonuclease fluorescence assay.

The present invention also provides a method as defined above, wherein the drug therapy comprises a drug that interacts with the dopamine D2 receptor. In a preferred embodiment, the drug therapy is clozapine therapy or a combination therapy comprising clozapine.

Also contemplated by the present invention is a method as defined above wherein the antipsychotic response comprises an improvement in one or more positive symptoms. Without wishing to be considered limiting in any manner, the positive symptoms may comprise delusions, hallucinations, disorganized speech, disorganized behavior, catatonic behavior or a combination thereof.

The present invention also contemplates a method as defined above wherein the subject is a schizophrenic subject.

Also provided by the present invention is a method of predicting antipsychotic response of an African American schizophrenic subject to clozapine therapy comprising,

testing a sample obtained from the subject for the presence of a polymorphism in the dopamine D2 receptor gene DRD2, wherein the presence of the rs1079598T allele, the rs1125394A allele or both indicates that the subject is susceptible to clozapine therapy.

Also contemplated by the present invention is a method of identifying susceptibility of a schizophrenic subject to drug therapy comprising:

testing a sample obtained from the subject for the presence of a polymorphism in the dopamine D2 receptor gene DRD2, wherein the presence of the rs1079598T allele, the rs1125394A allele or both indicates that the subject is susceptible to a drug therapy.

The present invention also provides a method as defined above wherein the presence of the rs1079598T allele and the rs1125394A allele indicates that the subject is susceptible to drug therapy.

In a further embodiment of the present invention as defined above, the subject is an African American subject.

Also contemplated by the method of the present invention as defined above, the sample obtained from the subject from comprise any sample from which DNA may be obtained, for example, but not limited to any biological fluid or tissue comprising DNA from the subject. In a preferred embodiment, the sample is blood.

The present invention also provides a method as defined above wherein the step of testing comprises DNA extraction. Further, any method known in the art may be employed to identify and or confirm one or more single nucleotide polymorphisms in the dopamine D2 receptor genes, for example, but not limited to PCR analysis, sequencing, 5′exonuclease fluorescence assay, probe hybridization or any combination thereof.

Also contemplated by the present invention is a method as defined above wherein the drug therapy comprises clozapine therapy or a combination therapy comprising clozapine.

In the method of the present invention as defined above, it is contemplated that the drug therapy, for example, but not limited to clozapine therapy or combination clozapine therapy results in improvement or amelioration of positive symptoms as described above.

In an alternate embodiment, there is provided a method of identifying susceptibility of an African American schizophrenic subject to clozapine therapy comprising:

testing a sample obtained from the subject for the presence of a polymorphism in the dopamine D2 receptor gene DRD2, wherein the presence of the rs1079598T allele, the rs1125394A allele or both indicates that the subject is susceptible to clozapine therapy.

The present invention examines uninvestigated regions of DRD2, and the effect of two intron 1 polymorphisms: rs1125394 A/G and rs1079598 C/F, on overall response, as well as positive and negative symptom response to clozapine in an African American patient population. The rs1079598 C/T polymorphism is only 12 base pairs downstream, towards the 3′ end of the gene from the TaqIB polymorphic site that has been associated with reduced D2 density in the human striatum (specifically the B1 allele) (Jonsson et al. 1999; Ritchie and Noble 2003). These data suggest that the rs1079598 C/T is important in D2 receptor function, and that variants in the DA D₂ gene (DRD2) might account for variability in patient response to clozapine, as well as other APDs.

Two dopamine D2 gene variants were analysed along with quantitative measures of positive and negative symptom response following clozapine treatment in an African American population. Experimental Procedures included African American schizophrenia patients who were genotyped by 5′-exonuclease fluorescence assays. Genotype groups were compared on the Brief Psychiatric Rating Scale (BPRS) overall score, and positive (BPOS) and negative symptom subscales (BNEG) using analysis of variance. In African Americans, the rs1079598 T allele and rs1125394 A allele, and a two-marker haplotype containing these two alleles were associated with improvement in overall BPRS and BPOS response. This study suggests that these D2 receptor gene variants provide significant prediction of antipsychotic response.

This summary of the invention does not necessarily describe all features of the invention.

### BRIEF DESCRIPTION OF THE DRAWINGS

These and other features of the invention will become more apparent from the following description in which reference is made to the appended drawings wherein:

FIG. 1 shows relative locations of DRD2 SNPs. Boxes represent exons; horizontal lines connecting boxes represent introns, promoter and untranslated regions; arrows indicate relative locations of SNPs.

### DETAILED DESCRIPTION

The following description is of a preferred embodiment.

The present invention examines the effect of two SNPs in the intron 1 region of DRD2 on clozapine response in human populations, for example but not limited to an African American schizophrenic patient population. In the African American sample, the rs1125394 A allele and rs1079598 T allele were associated with improvement in overall response on the brief psychiatric rating scale (BPRS) and positive symptom subscales (BPOS). The two-marker haplotype containing these two alleles (haplotype 1-2 or A-T) was found to be highly significantly associated with both of these response measures in human populations, for example, but not limited to African Americans.

In an embodiment of the present invention, the flanking sequences for the rs1125394 allele comprise:

TCATGTGCTTTGTATGAAAC[A/G]CCTTGGAATGCTGATAAGTTTAATT (SEQ ID NO:1) wherein [A/G] denotes either A or G at the position indicated. Similarly, the flanking sequences for the rs1079598 allele comprise:
GAATCACCTATTCYAAAGGCGAATC [C/T]GATCATGTGGTTCCTGCTGCCCTTA (SEQ ID NO:2) wherein [C/T] denotes either C or T at the position indicated. The method of the present invention also contemplates identifying one or more nucleotide sequences comprising SEQ ID NO: 1, SEQ ID NO:2, or both (comprising either polymorphism) in order to predict antipsychotic response to drug therapy. Similarly, the method of the present invention contemplates identifying nucleotide sequences that comprise fragments of either SEQ ID NO: 1, SEQ ID NO:2, or both that comprise the respective [A/G] or [C/T] sites. Preferably the fragments that are identified comprise at least 7 nucleotides including the polymorphic site, for example, 8, 9, 10, 11, 12, 13, 14, 15, 17, 19, 21, 25, 30, 35, 40, 45 or 50 consecutive nucleotides. The fragments may also be defined as comprising a range of nucleotides defined by any two of the values listed above.

The African American sample total BPRS response was significantly and positively correlated with BPOS improvement (Pearson's correlation (r)=0.693; p<0.001), while a small non-significant negative correlation was observed with negative symptoms (BNEG (r=−0.114; p=0.589)). This indicates that the significant findings for total BPRS are likely a result of clozapine's effects against positive symptoms in African Americans, but not negative symptoms.

One important aspect of this study is sample size. The current sample size allows an effect size of d=1.2 to be detected at an α-level of 0.05 with a power of 73% (Borenstein 2000), and d=1.5 to be detected with over 80% power.

Clozapine blood levels should also be discussed as this has been shown to be a predictor of therapeutic response. A blood clozapine threshold level of 350 ng/ml has been suggested to significantly increase the likelihood of clinical response in treatment refractory patients (Miller 1996). As mentioned in the Experimental Procedures section, clozapine blood levels were monitored throughout the course of treatment to ascertain compliance. The mean blood clozapine levels of a larger group of patients collected by Dr. Meltzer (n=97) was 399.10 ng/ml at the 6 month point of the study. Since the mean clozapine blood level of 399.10 ng/ml lies above the threshold of 350 ng/ml suggested by Miller (1996) the levels maintained in this study provide reasonable assurance that the large majority of patients were in the therapeutic range. Therefore, clozapine blood levels are not likely to contribute significantly to variation in response in this study.

In conclusion, we obtained evidence for two SNPs and a haplotype comprised of these two SNPs of the DRD2 gene to be associated with better overall BPRS score and positive symptoms improvement in human populations, for example, but not limited to African Americans.

According to an embodiment of the present invention, there is provided a method of predicting antipsychotic response to drug therapy comprising,

testing a sample obtained from a subject for the presence of a polymorphism in the dopamine D2 receptor gene DRD2, wherein the presence of the rs1079598T allele, the rs1125394A allele or both is predictive of the subject being susceptible to drug therapy.

The drug or combination of drugs employed in drug therapy comprises at least one drug that interacts with the dopamine D2 receptor. The drug or drugs may bind reversibly or irreversibly to the dopamine D2 receptor, or may act as competitive or non-competitive inhibitors to downregulate D2 receptor signalling. In a preferred embodiment, the drug employed in drug therapy is involved in dopamine D2 receptor blockade, more preferably the drug therapy comprises clozapine or a combination therapy comprising clozapine. However, any drug that interacts with the dopamine D2 receptor may be employed as a drug therapy alone or in combination with clozapine.

The presence of either the rs1079598T allele or the rs1125394A allele alone is predictive of the subject being susceptible to drug therapy, for example, but not limited to clozapine therapy, or a combination therapy comprising clozapine. In a further embodiment, the presence of both the rs1079598T and rs1125394A alleles is predictive of the subject being susceptible to drug therapy.

In an embodiment of the present invention, subjects from any ethnic race, age, gender or medical condition may be tested to determine if they are likely to be responsive to drug therapy. In this regard, a healthy subject or a subject that does not have any symptoms of a disease or medical condition may be tested to determine if he or she is likely to be responsive to drug therapy in the future if ever such therapy is needed. In this way, a proper course of treatment would be known in advance and progression of the disease could be stopped or slowed. Similarly, subjects that have been diagnosed with a disease such as, but not limited to schizophrenia may be tested to determine if a particular course of therapy is likely to bring about a therapeutic response.

As described above, but without wishing to be limiting in any manner the subject that is tested may comprise an individual with one or more psychotic symptoms, for example, but not limited to as described in DSM-IV which is hereby incorporated by reference. The psychotic symptoms may comprise positive symptoms such as, but not limited to distortions or exaggerations of inferential thinking (i.e. delusions), perception (i.e. hallucinations), language and communication (disorganised speech) and behavioral monitoring (grossly disorganized or catatonic behavior) or any combination thereof. Further, the positive symptoms may comprise distinct dimensions, for example, psychotic dimensions including, but not limited to delusions and hallucinations and disorganization dimensions including, but not limited to disorganized speech and behavior. It is also contemplated that the symptoms may comprise one or more negative symptoms, for example, but not limited to symptoms that reflect a dimunition or loss of normal function. Further, the subject may exhibit a combination of both positive and negative symptoms.

Although the method of the present invention may be employed to determine whether any subject is susceptible to drug therapy, in an embodiment of the present invention, which is not meant to be limiting in any manner, the subject that is tested is an African American subject. However, the method of the present invention may be employed on subjects of other races as described previously.

The sample obtained from the subject may comprise any tissue or biological fluid sample from which genomic DNA may be obtained. For example, but not wishing to be limiting, DNA may be obtained from blood, hair follicle cells, skin cells, cheek cells, tissue biopsy, or the like. In a preferred embodiment, the sample is blood.

The DNA of the subject may be tested for the presence or absence of one or both single nucleotide polymorphisms (SNPs) in their DNA by any suitable technique known in the art. Representative techniques that may be employed include without limitation PCR analysis, sequencing, 5′exonuclease fluorescence assay, probe hybridization or a combination thereof.

In a preferred embodiment of the present invention, the method as defined above is predictive of a subject's response to clozapine therapy or a combination therapy comprising clozapine. Further, it is contemplated that the antipsychotic response may comprise an improvement or amelioration in one or more positive symptoms, for example, but not limited to one or more positive symptoms including without limitation delusions, hallucinations, disorganized speech, disorganized behavior, catatonic behavior or a combination thereof. Without wishing to be limiting in any manner, the improvement may be determined by measuring a change in symptoms over the course of drug therapy in the Brief Psychiatric Rating Scale (BPRS), Positive symptom subscale (BPOS) or a combination thereof. For example, a subject that exhibits extreme catatonic behavior prior to treatment and no catatonic behavior following treatment is considered to exhibit an improvement in positive symptoms. It should also be noted that it is also possible to measure improvements using other scales/techniques of quantitative and/or qualitative measurements that are known in the art, and such scales and/or techniques may be employed in the method of the present invention.

In a preferred embodiment of the present invention which is not meant to be limiting in any manner, the subject is a schizophrenic subject.

In still a further embodiment of the present invention, which is not meant to be limiting in any manner, there is provided a method of predicting antipsychotic response of an African American schizophrenic subject to clozapine therapy comprising,

testing a sample obtained from the subject for the presence of a polymorphism in the dopamine D2 receptor gene DRD2, wherein the presence of the rs1079598T allele, the rs1125394A allele or both indicates that the subject is susceptible to clozapine therapy.

According to an alternate embodiment of the present invention which is not to be considered limiting in any manner, there is provided a method of identifying the susceptibility of a schizophrenic subject to drug therapy comprising:

testing a sample obtained from the subject for the presence of a polymorphism in the dopamine D2 receptor gene DRD2, wherein the presence of the rs1079598T allele, the rs1125394A allele or both indicates that the subject is susceptible to drug therapy.

In an alternate embodiment of the present invention, which is not meant to be limiting in any manner, there is provided a method of identifying the susceptibility of an African American schizophrenic subject to clozapine therapy comprising:

testing a sample obtained from the subject for the presence of a polymorphism in the dopamine D2 receptor gene DRD2, wherein the presence of the rs1079598T allele, the rs1125394A allele or both indicates that the subject is susceptible to clozapine therapy.

The present invention will be further illustrated in the following examples.

### EXAMPLES

When studying antipsychotic drug (APD) response it is useful to make a distinction between the positive and negative symptoms of schizophrenia (SCZ). This is because APD blockade of D₂ receptors in the mesolimbic pathway is thought to mediate the improvement of positive symptoms while the same action in the mesocortical pathway has been postulated to worsen negative symptoms (Stahl 2003). These components of treatment response are examined separately using a quantitative response measure (percentage (%) change).

### Clinical Sample

Clinical data were collected from 31 African American patients with a DSM-III-R or DSM-IV diagnoses of schizophrenia (APA 1994) were obtained at the Case Western Reserve University in Cleveland, Ohio (clinical trial coordinated by Dr. Meltzer). See Table I for sample characteristics.

TABLE 1
Characteristics of African American sample.

| | Mean BPRS | Mean BPOS | Mean BNEG | | |
|---|---|---|---|---|---|
| | Change ± | Change ± | Change ± | | |
| | SD/Mean Baseline | SD/Mean Baseline | SD/Mean Baseline | Males/ | Mean Age ± |
| | Score ± SD (n) | Score ± SD (n) | Score ± SD (n) | Females [n (%)] | SD |
| Afr.-Am. (n = 31) | −0.15 ± 0.30/ | −0.24 ± 0.30/ | 0.23 ± 1.33/ | 21/10(68/32) | 34.9 ± 10.9 |
| | 36.79 ± 14.22 (31) | 13.63 ± 5.22 (27) | 6.72 ± 4.52 (25) | | |

After informed consent was obtained patients underwent a wash-out period of 2 to 4 weeks during which, unless clinically necessary, they received no medications before starting clozapine. BPRS baseline ratings were obtained prior to starting clozapine treatment. Clozapine treatment was continued for a minimum of 6 months during which patients were evaluated prospectively. Clozapine blood levels were monitored throughout the course of treatment to ascertain compliance. Treatment response was evaluated as a % score change using the 18 item Brief Psychiatric Rating Scale (BPRS), a four item (conceptual disorganization, suspiciousness, hallucinations, unusual thought content) positive symptom subscale (BPOS) and a three item (emotional withdrawal, motor retardation, blunted affect) negative symptom subscale (BNEG) after 6 months of clozapine treatment from enrollment into the study (baseline) (with a negative value indicating an improvement in symptoms). Thus, % Score Change=(6 Month Score−Baseline Score)/(Baseline Score).

### Laboratory Methods

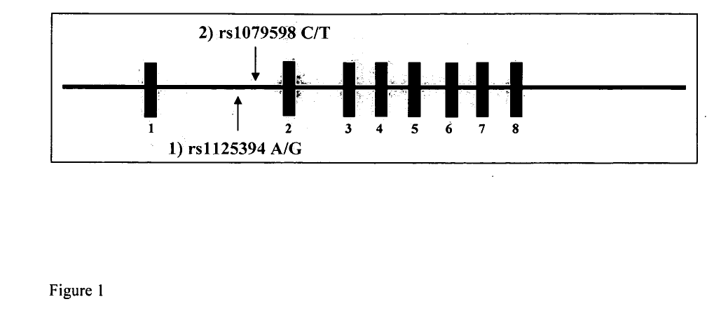

Blood samples were collected from the clinical site and sent to the Centre for Addiction and Mental Health (CAMH), Clarke site in Toronto, Ontario, Canada. Genomic DNA was extracted from whole blood using the high-salt method (Lahiri and Nurnberger 1991). Genotyping of the patients' DNA was performed at CAMH, Clarke site and the laboratory staff was blind to the psychiatric ratings. Genotyping was performed by 5′-exonuclease fluorescence assay (Shi et al. 1999) and results were confirmed by two researchers. We developed two SNP assays for DRD2 genotyping. From the 5′ end to the 3′ end of DRD2 the two SNPs were: 1. rs1125394 A/G; 2. rs1079598 C/T. The marker locations are shown in FIG. 1. Ambiguous genotypes were retyped until clear results were determined. Genotypes which did not produce clear results were excluded from further analyses.

### Statistical Methods

Individual SNP analyses of % score changes (continuous data) were performed using Analysis of Variance (ANOVA) in the Statistical Package for the Social Sciences, version 10.0.7 (SPSS 2000). Pairwise linkage disequilibrium was assessed using Haploview, version 2.04 (Barrett 2003). Haplotype analysis of unphased quantitative data was performed using QTPHASE (Dudbridge 2003). No significant deviation from Hardy-Weinberg equilibrium was observed for either SNP studied. Possible confounding factors of age and gender did not have a significant effect on treatment outcome measures either. As well, a high degree of linkage disequilibrium between the two SNPs was observed: D′=1.00, 95% confidence interval=0.44-1.00.

Individual SNP Associations with Clozapine Treatment Response

BPRS, BPOS and BNEG % change score distributions of genotype groups were compared against each other for the two SNPs. An allele present/absent (+/−) analysis was also performed for the rs1079598 SNP based on its close proximity to the TaqIB polymorphic site (see Introduction).

In our sample, both SNPs were significantly associated with treatment response (see Table 2).

TABLE 2
6 month change score comparisons among genotype groups (African American).

| | | African American | | | | | |
|---|---|---|---|---|---|---|---|
| | | BPRS | | | BPOS | | |
| | | Mean | | | Mean | | |
| SNP | Geno | Change (N) | 95% C.I.; S.D. | P | Change (N) | 95% C.I.; S.D. | P |
| | | *−0.15 (31) | −0.27/−0.04; 0.3 | | *−0.24 (27) | −0.36/−0.12; 0.3 | |
| rs1125394 | 11 | −0.24 (21) | −0.36/−0.13; 0.25 | <0.001 | −0.31 (19) | −0.45/−0.17; 0.29 | 0.030 |
| 1 = A | 12 | −0.09 (6) | −0.21/0.04; 0.12 | | −0.09 (5) | −0.35/0.18; 0.21 | |
| 2 = G | 22 | 0.60 (2) | −2.19/3.39; 0.31 | | 0.40 (1) | | |
| rs1079598 | 11 | — | — | 0.025 | — | — | 0.014 |
| 1 = C | 12 | 0.17 (4) | −0.53/0.86; 0.44 | | 0.16 (3) | −0.37/0.69; 0.21 | |
| 2 = T | 22 | −0.20 (25) | −0.31/−0.09; 0.27 | | −0.29 (22) | −0.42/−0.17; 0.28 | |
| | 1+ | 0.17 (4) | −0.53/0.86; 0.44 | 0.025 | 0.16 (3) | −0.37/0.69; 0.21 | 0.014 |
| | 1− | −0.20 (25) | −0.31/−0.09; 0.27 | | −0.29 (22) | −0.42/−0.17; 0.28 | |

Allele +/− genotype groups, e.g. 1+/1−, indicate genotype groups based on the presence (+) or absence (−) of a specified allele (1); asterisked (*) numbers indicate calculated values for the entire African-American sample. The change scores are written in proportions, such that a proportion score of −.24 is the same as a percentage change of −24%.

The rs1079598 C allele (−) was associated with better response than C allele (+) for both BPRS (p=0.025; Effect Size (d)=−1.22, 95% Confidence Interval (CI): −2.33/−0.12) and BPOS (p=0.014; d=−1.58, 95% CI: −2.87/−0.30). There was an allele dosage effect for allele 1 of the rs1125394 SNP and improvement in response for both BPRS (p<0.001) and BPOS (p=0.030). The effect size of the 1-1 genotype was d=−1.18 (95% CI: −2.05/−0.31; p=0.008) for the BPRS and d=−1.01 (95% CI: −1.97/−0.05; p=0.031) for the BPOS.

### Haplotype-Based Association

These two SNPs (rs1125394 and rs1079598) were analyzed together as a haplotype (see Table 3). This was done in order to determine if the SNPs would have greater predictive value when analyzed together.

TABLE 3
Haplotype analyses of rs1125394 and rs1079598.

| | African American | | | | | | | |
|---|---|---|---|---|---|---|---|---|
| | BPRS | | | | BPOS | | | |
| | Global | | Mean Change | Haplo | Global | | Mean Change | Haplo |
| Haplotype | P-Val. | Haplo | (N, Freq) | P-Val. | P-Val. | Haplo | (N, Freq) | P-Val. |
| | | | *−0.15 | | | | *−0.24 | |
| rs1125394/ | <0.001 | 1-2 | −0.22 (48, 0.83) | <0.001 | 0.005 | 1-2 | −0.29 (43, 0.86) | 0.002 |
| rs1079598 | | 2-2 | 0.20 (6, 0.10) | 0.008 | | 2-1 | 0.16 (3, 0.06) | 0.009 |

Asterisked (*) numbers indicate calculated values for the entire African American sample

This haplotype set had a global significance level of p<0.001 for the BPRS and p=0.005 for the BPOS. Specifically, haplotype 1-2 was associated with better response for both overall BPRS (p<0.001; d=−1.50, 95% CI: −2.23/−0.76; freq.=83%) and BPOS (p=0.002; d=−1.20, 95% CI: −2.03/−0.36; freq.=86%).

### REFERENCES

A.P.A. (1994) Diagnostic and Statistical Manual of Mental Disorders, 4^{th} ed. (DSM-IV). American Psychiatric Association, Washington, D.C.
Barrett J (2003) Haploview, version 2.04: See: broad.mit.edu/personal/jcbarret/haploview/
Borenstein, M. (2000) SamplePower, Release 2.0.
Carlsson, A. (1978) Antipsychotic drugs, neurotransmitters, and schizophrenia. Am. J. Psychiatry 135, 165-173.
Dubertret, C., Gouya, L., Hanoun, N., Deybach, J.-C., Ades, J., Hamon, M. and
Gorwood, P. (2004) The 3′ region of the DRD2 gene is involved in genetic susceptibility to schizophrenia. Schizophr. Res. 67, 75-85.
Dudbridge, F. (2003) Pedigree disequilibrium tests for multilocus haplotypes. Genet. Epidemiol. 25, 115-121.
Jonsson, E. G., Nothen, M. M., Grunhage, F., Farde, L., Nakashima, Y., Propping, P. and Sedvall, G. C. (1999) Polymorphisms in the dopamine D2 receptor gene and their relationships to striatal dopamine receptor density of healthy volunteers. Mol. Psychiatry 4, 290-296.
Lahiri, D. K. and Nurnberger, J. I. Jr. (1991) A rapid non-enzymatic method for the preparation of HMV DNA from blood for RFLP analysis. Nucleic Acids Res. 19, 5444.
Malhotra, A. K., Buchanan, R. W. and Kim, S. (1999) Allelic variation in the promoter region of the dopamine D2 receptor gene and clozapine response. Schizophr. Res. 36, 92-93.
Meltzer, H. Y. (1997) Treatment-resistant schizophrenia—the role of clozapine. Curr. Med. Res. Opin. 14, 1-20.
Meltzer, H. Y., Li, Z., Kaneda, Y. and Ichikawa, J. (2003) Serotonin receptors: their key role in drugs to treat schizophrenia. Prog. Neuropsychopharmacol. Biol. Psychiatry 27, 1159-1172.
Miller, D. D. (1996) The clinical use of clozapine plasma concentrations in the management of treatment-refractory schizophrenia. Ann Clin Psychiatry 8:99-109.
Neville, M. J., Johnstone, E. C. and Walton, R. T. (2004) Identification and characterization of ANKK1: a novel kinase gene closely linked to DRD2 on chromosome band 11q23.1. Hum. Mutat. 23, 540-545.
Noble, E. P. (1998) The D2 dopamine receptor gene: a review of association studies in alcoholism and phenotypes. Alcohol 1, 33-45.
Ohara, K., Nagai, M., Tani, K., Nakamura, Y., Ino, A. and Ohara, K. (1998) Functional polymorphism of −141C Ins/Del in the dopamine D2 receptor gene promoter and schizophrenia. Psychiatr. Res. 81, 117-123.
Pohjalainen, T., Rinne, J. O., Nagren, K., Lehikoinen, P., Anttila, K., Syvalahti, E. K. G. and Hietala, J. (1998) The A1 allele of the human D2 dopamine receptor gene predicts low D2 receptor availability in healthy volunteers. Mol. Psychiatry 3, 256-260.
Pohjalainen, T., Nagren, K., Syvalahti, E. K. G. and Hietala, J. (1999) The dopamine D2 receptor 5′-flanking variant, −141C Ins/Del, is not associated with reduced dopamine D2 receptor density in vivo. Pharmacogenetics 9, 505-509.
Ritchie, T., Noble, E. P. (2003) Association of seven polymorphisms of the D2 dopamine receptor gene with brain receptor-binding characteristics. Neurochem. Res. 28, 73-82.
Schafer, M., Rujescu, D., Giegling, I., Guntermann, A., Erfurth, A., Bondy, B. and Moller, H.-J. (2001) Association of short-term response to haloperidol treatment with a polymorphism in the dopamine D2 receptor gene. Am. J. Psychiatry 158, 802-804.
Shi, M. M., Myrand, S. P., Bleavins, M. R. and de la Iglesia, F. A. (1999) High throughput genotyping for the detection of a single nucleotide polymorphism in NAD(P)H quinine oxidoreductase (DT diaphorase) using TaqMan probes. Mol. Pathol. 52, 295-299.
Stahl, S. (2003) Key issues and unmet needs in schizophrenia. In: Kapur, S., and Lecrubier, Y. (Eds.), Dopamine in the pathophysiology and treatment of schizophrenia. Martin Dunitz, Taylor and Francis Group, London, New York, pp. 237-253.
Statistical Package for the Social Sciences (2000) S.P.S.S. for Windows, Rel. 10.0.7. S.P.S.S. Inc., Chicago.
Suzuki, A., Mihara, K., Kondo, T., Tanaka, O., Nagashima, U., Otani, K. and Kaneko, S. (2000) The relationship between dopamine D2 receptor polymorphism at the Taq1A locus and therapeutic response to nemonapride, a selective dopamine antagonist, in schizophrenic patients. Pharmacogenetics 10, 335-341.
Suzuki, A., Kondo, T., Mihara, K., Yasui-Furukori, N., Otani, K., Furukori, H, Kaneko, S. and Inoue, Y. (2001) Association between TaqIA dopamine D2 receptor polymorphism and therapeutic response to bromperidol: a preliminary report. Eur. Arch. Psychiatry Clin. Neurosci. 251, 57-59.
Thompson, J., Thomas, N., Singleton, A., Piggott, M., Lloyd, S., Perry, E. K., Morris, C. M., Perry, R. H., Ferrier, I. N. and Court, J. A. (1997) D2 dopamine receptor gene (DRD2) TaqIA polymorphism: reduced dopamine D2 receptor binding in the human striatum associated with the A1 allele. Pharmacogenetics 7, 479-484.

All citations are hereby incorporated by reference.

The present invention has been described with regard to one or more embodiments. However, it will be apparent to persons skilled in the art that a number of variations and modifications can be made without departing from the scope of the invention as defined in the claims.

What is claimed is:

1. A method of predicting antipsychotic response to drug therapy comprising,
testing a sample obtained from a subject for the presence of a polymorphism in the dopamine D2 receptor gene DRD2, wherein the presence of the rs1079598T allele, the rs1125394A allele or both is predictive of the subject being susceptible to drug therapy.

2. The method of claim 1, wherein the presence of the rs1079598T allele and the rs1125394A allele is predictive of the subject being susceptible to drug therapy.

3. The method of claim 1, wherein the subject is an African American subject.

4. The method of claim 1, wherein the sample is blood.

5. The method of claim 1, wherein the step of testing comprises DNA extraction and PCR analysis, sequencing, 5′exonuclease fluorescence assay, or a combination thereof.

6. The method of claim 1, wherein drug therapy comprises a drug that blocks the dopamine D2 receptor.

7. The method of claim 6, wherein the drug therapy is clozapine therapy.

8. The method of claim 1, wherein the antipsychotic response comprises an improvement in one or more positive symptoms.

9. The method of claim 8, wherein the one or more positive symptoms comprise delusions, hallucinations, disorganized speech, disorganized behavior, catatonic behavior or a combination thereof.

10. The method of claim 1, wherein the subject is a schizophrenic subject.

11. A method of predicting antipsychotic response of an African American schizophrenic subjects to clozapine therapy comprising,
testing a sample obtained from the subject for the presence of a polymorphism in the dopamine D2 receptor gene DRD2, wherein the presence of the rs1079598T allele, the rs1125394A allele or both indicates that the subject is susceptible to clozapine therapy.

12. A method of identifying the susceptibility of a schizophrenic subject to drug therapy comprising:
testing a sample obtained from the subject for the presence of a polymorphism in the dopamine D2 receptor gene DRD2, wherein the presence of the rs1079598T allele, the rs1125394A allele or both indicates that the subject is susceptible to a drug therapy.

13. The method of claim 12, wherein the presence of the rs1079598T allele and the rs1125394A allele indicates that the subject is susceptible to drug therapy.

14. The method of claim 12, wherein the subject is an African American subject.

15. The method of claim 12, wherein the sample is blood.

16. The method of claim 12, wherein the step of testing comprises DNA extraction and PCR analysis, sequencing, 5′exonuclease fluorescence assay, or a combination thereof.

17. The method of claim 12, wherein drug therapy comprises a drug that interacts with the dopamine D2 receptor.

18. The method of claim 17, wherein the drug therapy comprises clozapine therapy.

19. The method of claim 12, wherein said drug therapy results improvement in positive symptoms.

20. A method of identifying susceptibility of an African American schizophrenic subject to clozapine therapy comprising:
testing a sample obtained from the subject for the presence of a polymorphism in the dopamine D2 receptor gene DRD2, wherein the presence of the rs1079598T allele, the rs1125394A allele or both indicates that the subject is susceptible to clozapine therapy.
